(19) United States
(12) Patent Application Publication
(10) Pub. No.: US 2015/0335548 A1
(43) Pub. Date: Nov. 26, 2015

(54) Oral Care Composition

(71) Applicant: COLGATE-PALMOLIVE COMPANY, New York, NY (US)

(72) Inventors: Madhusudan Patel, Somerset, NJ (US); Mahmoud Hassan, Somerset, NJ (US)

(73) Assignee: Colgate-Palmolive Company, New York, NY (US)

(21) Appl. No.: 14/654,380

(22) PCT Filed: Dec. 20, 2012
(86) PCT No.: PCT/US12/70959

§ 371 Date: Jun. 19, 2015

(51) Int. Cl. A61K 8/41; A61K 8/49; A61K 8/46; A61Q 11/00; A61K 8/368

(52) U.S. Cl. CPC A61K 8/416; A61Q 11/00; A61K 8/368; A61K 8/466; A61K 8/494; A61K 8/4946; A61K 2800/30

(57) ABSTRACT

An oral care composition is provided which comprises a therapeutically effective amount of at least one ionic liquid for use in removing or reducing plaque, inhibiting the amount of bacteria in an oral cavity, inhibiting biofilm formation and/or degradation in an oral cavity and teeth whitening.

### BACKGROUND OF THE INVENTION

An ionic liquid is a class of salt comprising a cation and an anion that is in liquid at a temperature of 100° C. or less and commonly have melting points below room temperature. While not wishing to be bound by theory, ionic liquids generally have much lower symmetry than conventional salts and the charge of cation and anion is distrubuted over a larger volume of the molecule by resonance in ionic liquids which is thought to contribute to their liquid state at much lower temperatures than conventional salts (e.g. NaCl, mp 801° C.). Ionic liquids are often composed of a cation comprising a heterocyclic ring and a counter anion, often inorganic in nature. The nature of the cation and anion will determine the hydrophobicity, viscosity, density and other physical parameters and properties of the ionic liquid.

Ionic liquids have been evaluated as environmentally-friendly or ‘green’ alternatives to conventional organic solvents for a wide range of organic synthetic applications. Ionic liquids have unique characteristics that distinguish them from conventional organic solvents. For example, ionic liquids are non-volatile (i.e. they do not evaporate readily into the atmosphere), they have a high polarity and charge density, they may be hydrophobic or hydrophilic, and they have unique solvating properties. As such, ionic liquids are known to be used in cleaning compositions (for example, as disclosed in US 2006/0090777 A1 and U.S. Pat. No. 7,939,485 B2). A range of ionic liquids are commercially available, or they may be readily synthesized by simple ion-exchange reactions.

A biofilm is a structured group of microorganisms encapsulated within a self-developed polymeric extracellular matrix. Biofilms are typically adhered to a living or inert surface. In the human or animal body biofilms can form on any internal or external surface. Biofilms have been found to be involved in a wide variety of microbial infections in the body and cause a number of conditions including urinary tract infections, middle-ear infections, and in particular, diseases of the oral cavity.

Dental plaque is formed from a biofilm precursor, and is present to some degree on virtually all dental surfaces whether in the oral cavity or on dental instruments used by dental professionals. It comprises a dense microbial layer consisting of a mass of microorganisms embedded in a polysaccharide matrix. Plaque may form on any part of the tooth surface, and is found particularly at the gingival margin, and in cracks in the enamel. The danger associated with the formation of plaque on the teeth lies in the tendency of plaque to build up and eventually produce gingivitis, periodontitis and other types of periodontal disease, as well as dental caries and dental calculus. Dental plaque formation is also related to the feeling of a fuzzy tongue in an unclean oral cavity and as such addressing dental plaque formation can address cleaning the tongue.

Plaque itself adheres very firmly to dental surfaces and rapidly reforms on the tooth surface after it is removed. Current plaque removal methods rely primarily on the mechanical removal of plaque. These methods, which include brushing, brushing with an abrasive toothpaste, flossing, using interdental cleaners, scraping, using sonic energy (e.g. Sonicare toothbrushes) and ultrasound (e.g. Ultreo toothbrushes), in part, rely on a good brushing or flossing technique which many consumers simply do not possess. Moreover, these methods are particularly inefficient in removing stubborn plaque, or plaque hidden deep within cavities and fissures of teeth, between teeth or within gum pockets.

It is also known in the art to incorporate antimicrobial agents in oral compositions which destroy or retard the growth of bacteria. However, bacteria present in a biofilm or plaque deposit exhibit increased resistance to antimicrobial agents because the dense extracellular matrix and the outer layer of cells protect the bacteria found in the interior of the deposit from the effects of the antimicrobial agents.

There is therefore the need to provide improved methods and compositions for removing plaque which mitigate some of the inefficiencies resulting from a poor brushing/flossing technique and which effectively remove plaque hidden between teeth, within cavities and fissures of teeth, and in gum pockets.

It is also seen as being desirable by consumers to possess white teeth. Over time, teeth can darken or become stained. Teeth naturally become more yellow with age and can also be stained by food and drink. For example, teeth can be stained by tea, coffee, red wine and tobacco. Certain antibiotics, including for example tetracycline, can also stain teeth. Many consumers wish to whiten teeth to restore the natural colour and in some circumstances wish to whiten teeth beyond their natural colour.

It is known to use peroxide-based chemicals to whiten teeth. Carbamide peroxide can be used to bleach teeth. Carbamide peroxide generates hydrogen peroxide upon contact with water and the peroxide oxidizing agent bleaches stains. Whitening can be carried out by a dental professional, by a non-dental professional such as a beauty therapist or alternatively at home by the consumer themselves. Professional tooth whitening is expensive and time consuming.

Teeth can also be whitened and stains removed using abrasive agents to physically remove stains from the teeth. Abrasive agents can include baking soda (sodium bicarbonate), silica, aluminium oxide, calcium carbonate and calcium phosphate. However, abrasive agents can cause damage to the tooth enamel and can be especially damaging to softened tooth enamel that has become soft with age or with repeated attack with oral acids or the use of acidic foods and beverages. Furthermore, abrasive agents are not always suitable for whitening the teeth of those consumers who have dentures, crowns or who wear orthodontics. For these consumers, abrasive whitening agents can damage the ceramic surfaces or may not provide effective reach to clean between orthodontic wiring.

There is therefore the need to provide improved methods and compositions for whitening teeth which do not damage tooth enamel and which are suitable for use by consumers who have dentures, crowns or who wear orthodontics.

### SUMMARY OF THE INVENTION

The present invention aims at least partially to meet these needs in the art.

In a first aspect, the present invention provides an oral care composition comprising a therapeutically effective amount of at least one ionic liquid for use in removing or reducing plaque from the oral cavity of a subject.

Another embodiment of the invention is an oral care composition comprising a therapeutically effective amount of at least one ionic liquid for use in removing or reducing plaque from the oral cavity of a subject and an orally acceptable carrier for a mouth rinse, toothpaste, oral beads or strips, irrigation fluid, plaque removal liquid, tongue spray, dental floss, candy, lozenge, chewing gum, patches and lollipop.

Another embodiment of the oral care composition is where the ionic liquid comprises:
a) a heterocyclic cation; and
b) an anion selected from the group consisting of an anion selected from the group consisting of acetate, halide, phosphate, alkyl phosphate, phosphonate, pyrophosphate, hexametaphosphate, polymetaphosphate, orthophosphate, tripolyphosphate, sulfate, alkyl sulfate (e.g. methylsulfate, ethylsulfate), lauryl sulfate, phenolsulfate. benzoate, acetylacetonate, carboxylate, citrate, ascorbate, dicyamide, L- or D-amino acids (e.g. arginate, glycinate, prolinate, etc.), glycolate, gluconate, maleate, sweetener anions (e.g. saccharinate, aspartamate, cyclamate), hydroxide, succinate, tartrate, docusate, linoleate, oleate, and tosylate.

Another embodiment of the oral care composition is where the cation for the ionic liquid is selected from the group consisting of pyrrolium, pyrrolinium, pyrrolidinium, oxazolium, thiazolium, imidazolium, imidazolidinium, pyrazolium, pyrazolinium, pyrazolidinium, isoxazolium, isothiazolium, oxadiazolium, triazolium, thiadiazolium, pyridinium, piperidinium, morpholinium, thiomorphilinium, pyridazinium, pyrimidinium, pyrazinium, piperazinium, triazinium and quaternary ammonium.

Another embodiment of the oral care composition is where the cation for the ionic liquid is selected from the group consisting of imidazolium, pyrazolium and quaternary ammonium.

Optionally, the ionic liquid comprises:

a) an imidazolium cation, and
b) an anion selected from the group consisting of acetate, halide, phosphate, alkyl phosphate, sulfate, alkyl sulfate, and tosylate.

Preferably, the ionic liquid is selected from the group consisting of 1-ethyl-3-methylimidazolium (EMIM) chloride, EMIM bromide, EMIM ethyl sulfate, EMIM diethyl phosphate, EMIM acetate, EMIM tosylate, 1-butyl-3-methylimidazolium (BMIM) chloride, BMIM bromide, BMIM methyl sulfate, BMIM octyl sulfate, BMIM acetate, 1-allyl-3-methylimidazolium (AMIM) chloride, 1-decyl-3-methylimidazolium (DMIM) chloride, and 1,2,3-trimethylimidazolium (TMIM) methyl sulfate.

Optionally, the ionic liquid comprises:

a) a quaternary ammonium cation of the formula

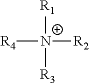

wherein R₁, R₂, R₃ and R₄ are each an organic moiety and may be the same or different, and at least one of R₁, R₂, R₃ and R₄ includes a hydroxyl, and

b) an anion selected from the group consisting of salicylate, alkylsulfate, sulfate, acetate, halide, phosphate, alkyl phosphate and tosylate.

Optionally, the quaternary ammonium cation is choline or tris-(2-hydroxyethyl)methylammonium.

Preferably, the ionic liquid is selected from the group consisting of choline salicylate, tris-(2-hydroxyethyl)methylammonium methylsulfate and mixtures thereof.

Optionally, the ionic liquid comprises:

a pyrazolium cation, and
an anion selected from the group consisting of acetate, halide, phosphate, alkyl phosphate, sulphate, alkyl sulphate and tosylate.

Preferably, the ionic liquid is 1,2,4-trimethylpyrazolium methylsulfate.

Optionally, the oral care composition is substantially free of any abrasives.

Further optionally, the ionic liquid is present in the composition in an amount of about 5 wt % to about 15 wt % based on the total weight of the composition. Still further optionally, the ionic liquid is present in the composition in an amount of about 8 wt % to about 10 wt % based on the total weight of the composition.

Optionally, the oral care composition comprises an orally acceptable carrier for a mouth rinse, toothpaste, oral beads or strips, irrigation fluid, plaque removal liquid, tongue spray, dental floss, candy, lozenge, chewing gum, patches (e.g. intra oral patch similar to smokeless tobacco pouches) and lollipop.

Further optionally, the composition further comprises one or more agents selected from selected from diluents, bicarbonate salts, pH modifying agents, surfactants, foam modulators, thickening agents, viscosity modifiers, humectants, sweeteners, flavorants, pigments, anticaries agents, anticalculus or tartar control agents, and mixtures thereof.

In a second aspect, the present invention provides an oral care composition comprising a therapeutically effective amount of at least one ionic liquid for use in whitening teeth in a subject.

Optionally, the oral care composition is as defined herein.

Further optionally, the composition is substantially free of any abrasives and/or peroxides.

In a third aspect, the present invention provides an oral care composition comprising a therapeutically effective amount of at least one ionic liquid for use in inhibiting biofilm formation and/or degrading biofilm in the oral cavity of a subject.

Optionally, the oral care composition is as defined herein.

In a fourth aspect, the present invention provides an oral care composition comprising a therapeutically effective amount of at least one ionic liquid for use in reducing the amount of bacteria in the oral cavity of a subject, wherein the oral care composition is selected from a mouth rinse, toothpaste, toothpowder, oral bead or strip, fluid-encased dental strip, irrigation fluid, plaque removal liquid, dental floss, hard candy, soft candy, lozenge, chewing gum, or lollipop. Optionally, the oral care composition is as defined herein.

In a fifth aspect, the present invention provides a method of removing or reducing plaque from the oral cavity of a subject comprising administering to the subject an oral care composition comprising a therapeutically effective amount of an ionic liquid.

Optionally, the oral care composition is as defined herein.

In a sixth aspect, the present invention provides a method of whitening teeth in a subject comprising administering to the subject an oral care composition comprising a therapeutically effective amount of an ionic liquid.

Optionally, the oral care composition is as defined herein.

In a seventh aspect, the present invention provides a method of inhibiting biofilm formation and/or degrading biofilm in the oral cavity of a subject comprising administering to the subject an oral care composition comprising a therapeutically effective amount of an ionic liquid.

Optionally, the oral care composition is as defined herein.

In an eighth aspect, the present invention provides a method of reducing the amount of bacteria in the oral cavity of a subject comprising administering to the subject an oral care composition comprising a therapeutically effective amount of an ionic liquid, wherein the oral care composition is selected from a mouth rinse, toothpaste, toothpowder, oral bead or strip, fluid-encased dental strip, irrigation fluid, plaque removal liquid, dental floss, hard candy, soft candy, lozenge, chewing gum, or lollipop.

Optionally, the oral care composition is as defined herein.

In a ninth aspect, the present invention provides a use of an ionic liquid, in an oral care composition, for removing or reducing plaque in the oral cavity of a subject.

In a tenth aspect, the present invention provides a use of an ionic liquid, in an oral care composition, for whitening teeth in a subject.

In an eleventh aspect, the present invention provides a use of an ionic liquid, in an oral care composition, for inhibiting biofilm formation, and/or degrading biofilm in the oral cavity of a subject.

In a twelfth aspect, the present invention provides a use of an ionic liquid, in an oral care composition, for reducing the amount of bacteria the oral cavity of a subject, wherein the oral care composition is selected from a mouth rinse, toothpaste, toothpowder, oral bead or strip, fluid-encased dental strip, irrigation fluid, plaque removal liquid, dental floss, hard candy, soft candy, lozenge, chewing gum, or lollipop.

Further embodiments of the invention will be apparent from the detailed description and the examples.

### DESCRIPTION OF THE INVENTION

It should be understood that the detailed description, and specific examples, while indicating embodiments of the invention, are intended for purposes of illustration only and are not intended to limit the scope of the invention.

As used throughout, ranges are used as shorthand for describing each and every value that is within the range. Any value within the range can be selected as the terminus of the range.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about,” when applied to the value for a parameter of a composition or method of this invention, indicates that the calculation or the measurement of the value allows some slight imprecision without having a substantial effect on the chemical or physical attributes of the composition or method. If, for some reason, the imprecision provided by “about” is not otherwise understood in the art with this ordinary meaning, then “about” as used herein indicates a possible variation of up to 5% in the value.

As referred to herein, all compositional percentages are by weight of the total composition, unless otherwise specified.

As used herein, the term “therapeutically effective amount” refers to an amount sufficient to bring about the desired therapeutic effect.

As used herein, the term “biofilm removal” is encompasses inhibition of biofilm formation, and biofilm degradation.

In some embodiments, the present invention provides an oral care composition comprising a therapeutically effective amount of at least one ionic liquid for use in removing or reducing plaque from the oral cavity of a subject.

In other embodiments, the present invention provides an oral care composition comprising a therapeutically effective amount of at least one ionic liquid for use in whitening teeth in a subject.

In further embodiments, the present invention provides an oral care composition comprising a therapeutically effective amount of at least one ionic liquid for use in inhibiting biofilm formation and/or degrading biofilm in the oral cavity of a subject.

In yet further embodiments, the present invention provides an oral care composition comprising a therapeutically effective amount of at least one ionic liquid for use in reducing the amount of bacteria in the oral cavity of a subject, wherein the oral care composition is selected from a mouth rinse, toothpaste, toothpowder, oral bead or strip, fluid-encased dental strip, irrigation fluid, plaque removal liquid, dental floss, hard candy, soft candy, lozenge, chewing gum, or lollipop.

### Ionic Liquid

The term “ionic liquid” used in the context of the present invention means a salt comprising a cation and an anion that is in liquid at a temperature of 100° C. or less and commonly have melting points below room temperature.

Any anion mentioned herein may be used in combination with any of the cation to form the oral care compositions for use according to the present invention.

As used herein, the term “alkyl” refers to a saturated aliphatic hydrocarbon, including straight-chain, branched-chain and cycloalkyl groups of 1 to 20 carbon atoms. Alkyl groups include methyl, ethyl, propyl, butyl, pentyl, hexyl, heptyl, octyl, nonyl and decyl.

As used herein, the term “cycloalkyl” refers to a C₃₋₈ cyclic hydrocarbon.

As used herein, the term “alkenyl” refers to an unsaturated, open chain hydrocarbon with 2 to 20 carbon atoms and with one or more carbon-carbon double bonds. For example, alkenyl groups include allyl and vinyl.

In one arrangement, the ionic liquid comprises:

a) a heterocyclic cation or a quaternary ammonium cation; and
b) an anion selected from the group consisting of an anion selected from the group consisting of acetate, halide, phosphate, alkyl phosphate, phosphonate, pyrophosphate, hexametaphosphate, polymetaphosphate, orthophosphate, tripolyphosphate, sulfate, alkyl sulfate (e.g. methylsulfate, ethylsulfate), lauryl sulfate, phenolsulfate. benzoate, acetylacetonate, carboxylate, citrate, ascorbate, dicyamide, L- or D-amino acids (e.g. arginate, glycinate, prolinate, etc.), glycolate, gluconate, maleate, sweetener anions (e.g. saccharinate, aspartamate, cyclamate), hydroxide, succinate, tartrate, docusate, linoleate, oleate, and tosylate.

In one arrangement, the heterocyclic cation is selected from the group consisting of pyrrolium, pyrrolinium, pyrrolidinium, oxazolium, thiazolium, imidazolium, imidazolidinium, pyrazolium, pyrazolinium, pyrazolidinium, isoxazolium, isothiazolium, oxadiazolium, triazolium, thiadiazolium, pyridinium, piperidinium, morpholinium, thiomorphilinium, pyridazinium, pyrimidinium, pyrazinium, piperazinium, triazinium and quaternary ammonium.

In one arrangement, the ionic liquid comprises:

a) a cation selected from the group consisting of an imidazolium cation, quaternary ammonium cation, a pyrazolium cation and mixtures thereof;
b) an anion selected from the group consisting of acetate, halide, phosphate, alkyl phosphate, phosphonate, pyrophosphate, hexametaphosphate, polymetaphosphate, orthophosphate, tripolyphoisphate, sulfate, alkyl sulfate (e.g. methylsulfate, ethylsulfate), lauryl sulfate, phenolsulfate. benzoate, acetylacetonate, carboxylate, citrate, ascorbate, dicyamide, L- or D-amino acids (e.g. arginate, glycinate, prolinate, etc.), glycolate, gluconate, maleate, sweetener anions (e.g. saccharinate, aspartamate, cyclamate), hydroxide, succinate, tartrate, docusate, linoleate, oleate and tosylate.

In one arrangement, the ionic liquid comprises:

a) an imidazolium cation, and
b) an anion selected from the group consisting of acetate, halide, phosphate, alkyl phosphate, sulfate, alkyl sulfate, and tosylate.

An “imidazolium” cation as used in the context of this application may optionally have substituents including H, alkyl, alkenyl or aryl at positions 1 to 5.

In some embodiments, the imidazolium ion has the formula 1-R¹-2-R²-3-R³-imidazolium, represented by the structure below, wherein R¹, R² and R³ are independently selected from H, alkyl and alkenyl.

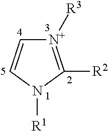

R¹, R² and R³ may be the same or different. The alkyl or alkenyl groups mentioned herein may be linear or branched. Typically, the alkyl or alkenyl groups are linear.

Optionally, R¹, R² and R³ are independently selected from H, C₁₋₂₂ alkyl and C₂₋₂₂ alkenyl. In another embodiment, R¹, R² and R³ are independently selected from H, C₁₂₋₂₂ alkyl and C₁₂₋₂₂ alkenyl. In still another embodiment, R¹, R² and R³ are independently selected from H, C₁₋₈ alkyl and C₂₋₈ alkenyl. In yet another embodiment, R¹, R² and R³ are independently selected from H, C₁₋₄ alkyl and C₂₋₄ alkenyl.

In some embodiments, R¹, R² and R³ are independently selected from H, C₁₋₁₀ alkyl and C₂₋₁₀ alkenyl. In a preferred embodiment, R¹ and R³ are independently selected from C₁₋₁₀ alkyl and C₂₋₁₀ alkenyl, and R² is independently selected from, H, C₁₋₁₀ alkyl and C₂₋₁₀ alkenyl.

In some embodiments, the C₁₋₁₀ alkyl and C₂₋₁₀ alkenyl are linear. In other embodiments, the C₁₋₁₀ alkyl and C₂₋₁₀ alkenyl are branched.

In some embodiments, R¹ is C₄₋₁₀ alkyl or C₄₋₁₀ alkenyl. In other embodiments, R¹ is C₆₋₁₀ alkyl or C₆₋₁₀ alkenyl. In yet further embodiments, R¹ is C₈₋₁₀ alkyl or C₈₋₁₀ alkenyl.

In a preferred embodiment, R³ is methyl.

Typically, R² is H. Optionally, R¹ is selected from C₁₋₁₀ alkyl, or from C₄₋₁₀ alkyl, or from C₆₋₁₀ alkyl, R² is H, and R³ is methyl. Alternatively, R¹ is selected from C₂₋₁₀ alkenyl, or from C₄₋₁₀ alkenyl, or from C₆₋₁₀ alkenyl, R² is H, and R³ is optionally methyl.

In some embodiments, R¹, R², and R³ are independently selected from H, C₄₋₁₀ alkyl and C₄₋₁₀ alkenyl, or from H, C₆₋₁₀ alkyl and C₆₋₁₀ alkenyl, or from H, C₈₋₁₀ alkyl and C₈₋₁₀ alkenyl.

In a preferred embodiment, R¹ is selected from C₄₋₁₀ alkyl and C₄₋₁₀ alkenyl, or from C₆₋₁₀ alkyl and C₆₋₁₀ alkenyl, or from C₈₋₁₀ alkyl and C₈₋₁₀ alkenyl, R² is H, and R³ is optionally methyl.

In one embodiment, R¹, R² and R³ are methyl.

In a typical embodiment, R¹ is a methyl, ethyl, propyl, butyl, or pentyl, R² is H, and R³ is methyl.

In another embodiment, R¹ is C₆₋₁₀ alkyl (hexyl, heptyl, octyl, nonyl or decyl), R² is H and R³ is methyl.

In yet another embodiment, R¹ is allyl, R² is H and R³ is methyl.

In a further embodiment, R¹ is vinyl, R² is H and R³ is methyl.

In some embodiments, R¹ and R² are independently selected from C₁₋₅ alkyl (methyl, ethyl, propyl, butyl, or pentyl), and R³ is methyl.

In other embodiments, R¹ and R² are independently selected from C₆₋₁₀ alkyl (hexyl, heptyl, octyl, nonyl or decyl), and R³ is methyl.

In yet further embodiments, R¹ is vinyl or allyl, R² is selected from methyl, ethyl, propyl, butyl, or pentyl, and R³ is methyl.

### Anions

As used herein, the term “halide” refers to F, Cl, Br, I. In some embodiments, the anion is a halide selected from Br and Cl.

As used herein, the term “alkyl” is as defined above.

In some embodiments, the anion is an alkyl sulfate selected from methyl sulfate, ethyl sulfate, propyl sulfate, butyl sulfate, pentyl sulfate, hexyl sulfate, heptyl sulfate, and octyl sulfate. In a preferred embodiment, the anion is octyl sulfate.

In some embodiments, the alkyl sulfate and alkyl phosphate comprise from 1 to 22 carbon atoms. Preferably, the alkyl sulfate and alkyl phosphate comprise from 1 to 4, from 6 to 10 carbon atoms or from 12 to 22 carbon atoms.

Typically, the anion is selected from the group consisting of acetate, bromide, chloride, methyl sulfate, ethyl sulfate, octyl sulfate, diethyl phosphate, and tosylate.

In preferred embodiments, the anion is selected from the group consisting of acetate, octylsulfate or tosylate.

In another embodiment, the anion is bromide.

In a further embodiment, anion is diethylphosphate.

In yet a further embodiment, the anion is tosylate.

In still yet a further embodiment, the anion is acetate.

In preferred embodiments, the ionic liquid is selected from 1-ethyl-3-methylimidazolium (EMIM) chloride, EMIM bromide, EMIM ethyl sulfate, EMIM diethyl phosphate, EMIM acetate, EMIM tosylate, 1-butyl-3-methylimidazolium (BMIM) chloride, BMIM bromide, BMIM methyl sulfate, BMIM octyl sulfate, BMIM acetate, 1-allyl-3-methylimidazolium (AMIM) chloride, 1-decyl-3-methylimidazolium (DMIM) chloride, and 1,2,3-trimethylimidazolium (TMIM) methyl sulfate.

In another arrangement, the ionic liquid comprises:

a) a quaternary ammonium cation of the formula

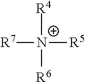

wherein R⁴, R⁵, R⁶ and R⁷ are each an organic moiety and may be the same or different, and optionally, wherein at least one of R⁴, R⁵, R⁶ and R⁷ includes a hydroxyl, and

b) an anion selected from the group consisting of salicylate, alkylsulfate, sulfate, acetate, halide, phosphate, alkyl phosphate and tosylate.

In some embodiments, R⁴, R⁵, R⁶ and R⁷ are independently selected from substituted or unsubstituted C₁₋₂₂ alkyl and C₂₋₂₂ alkenyl.

In some embodiments, R⁴, R⁵, R⁶ and R⁷ are independently selected from substituted or unsubstituted C₁₋₆ alkyl and C₂₋₆ alkenyl.

In some embodiments, at least one of R⁴, R⁵, R⁶ and R⁷ is a hydroxyalkyl group. In some embodiments, the hydroxyalkyl group is a C₁-C₆ hydroxyalkyl group, or a C₁-C₄ hydroxyalkyl group.

In some embodiments, the hydroxyalkyl group is a hydroxyethyl group. In some embodiments, the hydroxyalkyl group is a hydroxymethyl group.

In some embodiments, one of R⁴, R⁵, R⁶ and R⁷ is a hydroxyalkyl group and three of R⁴, R⁵, R⁶ and R⁷ are substituted or unsubstituted alkyl groups. In other embodiments, two of R⁴, R⁵, R⁶ and R⁷ are hydroxyalkyl groups and two of R⁴, R⁵, R⁶ and R⁷ are substituted or unsubstituted alkyl groups. In other embodiments, three of R⁴, R⁵, R⁶ and R⁷ are hydroxyalkyl groups and one of R⁴, R⁵, R⁶ and R⁷ is a substituted or unsubstituted alkyl group.

In some embodiments, the alkyl group is a substituted or unsubstituted C₁-C₆ alkyl group, or a substituted or unsubstituted C₁-C₄ alkyl group. In some embodiments, the alkyl group is an ethyl group. In some embodiments, the alkyl group is a methyl group

In some embodiments, the quaternary ammonium cation is choline or tris-(2-hydroxyethyl)methylammonium.

The anion may be as described above. Preferably, the anion is selected from methylsulfate and salicylate.

In preferred embodiments, the ionic liquid is choline salicylate or tris-(2-hydroxyethyl)methylammonium methylsulfate.

In another arrangement, the ionic liquid comprises:

a pyrazolium cation, and
an anion selected from the group consisting of acetate, halide, phosphate, alkyl phosphate, sulphate, alkyl sulphate and tosylate.

A “pyrazolium” cation as used in the context of the present invention, has the general basic ring structure of Formula 3, and optionally has substituents including H, alkyl, alkenyl or aryl at positions R₁ to R₅.

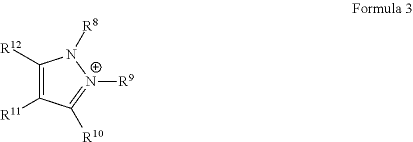

In some embodiments, R⁸, R⁹, R¹⁰, R¹¹ and R¹² are independently selected from H, alkyl and alkenyl.

In some embodiments, R⁸, R⁹, R¹⁰, R¹¹ and R¹² are independently selected from H, C₁₋₂₂ alkyl and C₂₋₂₂ alkenyl.

In some embodiments R⁸, R⁹, R¹⁰, R¹¹ and R¹² are the same or different and are independently selected from H and C₁ to C₄ alkyl groups.

In some embodiments, R¹⁰ and R¹² are H and R⁸, R⁹ and R¹¹ are independently selected from alkyl and alkenyl. R⁸, R⁹ and R¹¹ may be the same or different. Optionally, R¹⁰ and R¹² are H and R⁸, R⁹ and R¹¹ are C₁₋₄ alkyl.

In a preferred embodiment, R⁸, R⁹ and R¹¹ are methyl.

Typically, R¹⁰ and R¹² are H.

In a typical embodiment, R⁸, R⁹ and R¹¹ are methyl, ethyl, propyl, or butyl, and R₃ and R₅ are H.

The anion may be as described above. In a preferred embodiment, the anion is methyl sulfate.

Preferably, the ionic liquid is 1,2,4-trimethylpyrazolium methylsulfate.

Typically, the ionic liquid is present in the oral care composition in an amount of about 0.1 wt % to about 30 wt % based on the total weight of the composition.

In some embodiments, the ionic liquid is present in the oral care composition in an amount of about 0.5 wt % to about 20 wt %, or from about 1 wt % to about 15 wt %, based on the total weight of the composition.

Optionally, the ionic liquid is present in the oral care composition in an amount of about 5 wt % to about 15 wt %, or from about 7 wt % to about 12 wt %, based on the total weight of the composition.

Preferably, the ionic liquid is present in the oral care composition in an amount of about 8 wt % to about 10 wt % based on the total weight of the composition.

In some embodiments, the ionic liquid is present in the oral care composition in a concentration of about 1 mM to about 500 mM, or from about 40 mM to about 400 mM. Optionally, the ionic liquid is present in the oral care composition in a concentration of about 5 mM to about 300 mM or from about 10 mM to about 270 mM or from 1 mM to 50 mM.

Preferably, the ionic liquid is present in the oral care composition in a concentration of about 15 mM to about 250 mM, or from about 18 mM to about 220 mM.

### Abrasives

Whilst, the compositions for use according to the present invention may optionally further comprise an abrasive which may be useful, for example, as a polishing agent, it has been found that oral care compositions comprising ionic liquids as defined herein, are effective in removing biofilm or plaque, and whitening teeth, without the need for substantial amounts of abrasives. This is advantageous because abrasives can damage enamel and expose dentine tissues with repeated use, particularly, in subjects with soft enamel caused by disease or excessive exposure to food acids.

In one embodiment, the oral care composition comprises an abrasive in an amount of less than 0.1 wt % by total weight of the composition.

In another embodiment, the oral care composition comprises an abrasive in an amount of less than 0.01 wt % by total weight of the composition.

In yet another embodiment, the composition is substantially free, or free, of any abrasives.

Suitable optional abrasives include silica, for example in the form of precipitated silica or as admixed with alumina, insoluble phosphates, calcium carbonate, and mixtures thereof. Among insoluble phosphates useful as abrasives are orthophosphates, polymetaphosphates and pyrophosphates. Illustrative examples are dicalcium orthophosphate dihydrate, calcium pyrophosphate, calcium pyrophosphate, tricalcium phosphate, calcium polymetaphosphate and insoluble sodium polymetaphosphate.

### Carrier

Among useful carriers for optional inclusion in a composition for use according to the invention are diluents, bicarbonate salts, pH modifying agents, surfactants, foam modulators, thickening agents, viscosity modifiers, humectants, sweeteners, flavorants, pigments, anticaries agents, and anticalculus or tartar control agents. Carriers should be selected for compatibility with each other and with other ingredients of the composition.

Water is a preferred diluent and in some compositions such as mouthwashes, water is commonly accompanied by an alcohol, e.g., ethanol. The weight ratio of water to alcohol in a mouthwash composition is generally 1:1 to 20:1, for example 3:1 to 20:1 or 4:1 to 10:1. In a whitening liquid, the weight ratio of water to alcohol can be within or below the above ranges, for example, 1:10 to 2:1.

In a further embodiment, the composition for use according to the invention comprises at least one bicarbonate salt, useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. One or more bicarbonate salts are optionally present in a total amount of about 0.1 wt % to about 50 wt %, for example about 1 wt % to 20 wt %, by total weight of the composition.

In a still further embodiment, the composition for use according to the invention comprises at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like. One or more pH modifying agents are optionally present in a total amount effective to maintain the composition in an orally acceptable pH range.

In a still further embodiment, the composition for use according to the invention comprises at least one surfactant. Any orally acceptable surfactant, most of which are anionic, nonionic or amphoteric, can be used. Suitable anionic surfactants include without limitation, water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation, derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01 wt % to about 10 wt %, for example, from about 0.05 wt % to about 5 wt %, or from about 0.1 wt % to about 2 wt % by total weight of the composition.

In a still further embodiment, the composition for use according to the invention comprises at least one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000. One or more PEGs are optionally present in a total amount of about 0.1 wt % to about 10 wt %, for example from about 0.2 wt % to about 5 wt %, or from about 0.25 wt % to about 2 wt %, by total weight of the composition.

In a still further embodiment, the composition for use according to the invention comprises at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly t-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol® series. Particularly preferred Carbopols include Carbopol 934, 940, 941, 956, 974P, and mixtures thereof. One or more thickening agents are optionally present in a total amount of from about 0.01 wt % to 15 wt %, for example from about 0.1 wt % to about 10 wt %, or from about 0.2 wt % to about 5 wt %, by total weight of the composition.

In a still further embodiment, the composition for use according to the invention comprises at least one viscosity modifier, useful for example to inhibit settling or separation of ingredients or to promote re-dispersibility upon agitation of a liquid composition. Any orally acceptable viscosity modifier can be used, including without limitation, mineral oil, petrolatum, clays and organomodified clays, silica and the like. One or more viscosity modifiers are optionally present in a total amount of from about 0.01 wt % to about 10 wt %, for example, from about 0.1 wt % to about 5 wt %, by total weight of the composition.

In a still further embodiment, the composition for use according to the invention comprises at least one humectant. Any orally acceptable humectant can be used, including without limitation, polyhydric alcohols such as glycerin, sorbitol, xylitol or low molecular weight PEGs. Most humectants also function as sweeteners. One or more humectants are optionally present in a total amount of from about 1 wt % to about 70 wt %, for example, from about 1 wt % to about 50 wt %, from about 2 wt % to about 25 wt %, or from about 5 wt % to about 15 wt %, by total weight of the composition.

In a still further embodiment, a composition for use according to the invention comprises at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount depending strongly on the particular sweetener(s) selected, but typically 0.005 wt % to 5 wt %, by total weight of the composition.

In a still further embodiment, a composition for use according to the invention comprises at least one flavorant, useful for example to enhance taste of the composition. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt % to about 5 wt %, for example, from about 0.1 wt % to about 2.5 wt %, by total weight of the composition.

In a still further embodiment, a composition for use according to the invention may comprise at least one colorant. Colorants herein include pigments, dyes, lakes and agents imparting a particular luster or reflectivity such as pearling agents. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of from about 0.001 wt % to about 20 wt %, for example, from about 0.01 wt % to about 10 wt %, or from about 0.1 wt % to about 5 wt %, by total weight of the composition.

In some embodiments, the composition for use according to the invention comprises a fluoride ion source. Fluoride ion sources include, but are not limited to: stannous fluoride, sodium fluoride, potassium fluoride, potassium monofluorophosphate, sodium monofluorophosphate, ammonium monofluorophosphate, sodium fluorosilicate, ammonium fluorosilicate, amine fluoride such as olaflur (N′-octadecyltrimethylendiamine-N,N,N′-tris(2-ethanol)-dihydrofluoride), ammonium fluoride, and combinations thereof. In certain embodiments the fluoride ion source includes stannous fluoride, sodium fluoride, amine fluorides, sodium monofluorophosphate, as well as mixtures thereof. In certain embodiments, the oral care composition of the invention may also contain a source of fluoride ions or fluorine-providing ingredient in amounts sufficient to supply about 50 to about 5000 ppm fluoride ion, e.g., from about 100 to about 1000, from about 200 to about 500, or about 250 ppm fluoride ion. Fluoride ion sources may be added to the compositions of the invention at a level of about 0.001 wt % to about 10 wt %, e.g., from about 0.003 wt % to about 5 wt %, 0.01 wt % to about 1 wt, or about 0.05 wt %. However, it is to be understood that the weights of fluoride salts to provide the appropriate level of fluoride ion will obviously vary based on the weight of the counter ion in the salt, and one of skill in the art may readily determine such amounts. A preferred fluoride salt may be sodium fluoride.

The composition for use according to the present invention optionally comprises a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

The composition for use according to the present invention optionally incorporates one or more antisensitivity agents, e.g., potassium salts such as potassium nitrate, potassium bicarbonate, potassium chloride, potassium citrate, and potassium oxalate; capsaicin; eugenol; strontium salts; zinc salts; chloride salts and combinations thereof. Such agents may be added in effective amounts, e.g., from about 1 wt % to about 20 wt % by weight based on the total weight of the composition, depending on the agent chosen. The compositions of the present invention may also be used to treat hypersensitivity by blocking dentin tubules when applied to a tooth.

In some embodiments, the composition for use according to the invention further comprises an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

In another embodiment, the composition for use according to the invention comprises an orally acceptable zinc ion source useful, for example, as an antimicrobial, anticalculus or breath-freshening agent. One or more such sources can be present. Suitable zinc ion sources include without limitation zinc acetate, zinc citrate, zinc gluconate, zinc glycinate, zinc oxide, zinc sulfate, sodium zinc citrate and the like. One or more zinc ion sources are optionally and illustratively present in a total amount of from about 0.05 wt % to about 3 wt %, for example from about 0.1 wt % to about 1 wt %, by total weight of the composition.

The composition for use according to the present invention may additionally optionally comprise a tartar control (anticalculus) agent as provided below. Tartar control agents among those useful herein include salts of the specified agents, including alkali metal and ammonium salts. The agents include: phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof. Other useful tartar control agents include polycarboxylate polymers and polyvinyl methyl ether/maleic anhydride (PVM/MA) copolymers, such as GANTREZ®.

In some embodiments, the composition for use according to the present invention further comprises a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophan, L-lysine, methionine, threonine, levocarnitine and L-carnitine), lipotropics (such as choline, inositol, betaine, and linoleic acid), and mixtures thereof.

In some embodiments, the oral care composition of the invention does not contain any other antibacterial or whitening agent.

### Delivery

The oral care composition for use according to the present invention preferably comprises an orally acceptable carrier for use in a product such as a mouth rinse (including dual phase mouthwash), toothpaste, actives in beads/strips, irrigation fluids, plaque removal fluids, Wisp® formulas, formulations to be delivered through devices such as pens, back of a toothbrush and front of a toothbrush, formulations to be delivered through porous wicking materials, interdental brushes, fluid encased dental strips, floss impregnated or coated with the formulations or dried formulations, portables, oral trays, hard or soft candy, lozenge with a soft plaque dissolving liquid inside, lollipops with the plaque dissolving formulation imbedded into the lickable “candy” that can also help control tongue bacteria, peelable gels, patches (e.g. intra oral patch similar to smokeless tobacco pouches), formulations for pop-rocks that upon popping, spread a fine mist of the formulation around oral cavity, tongue cleaners with plaque dissolving strips and dental strips. Accordingly, opportunities exist for professional use of the compositions of the present invention (e.g. during cleanings, irrigations, or aggressive periodontal procedures, such as root planning & scaling). If used in animals or pets, veterinary pastes, chewables or treats may also be used as the orally acceptable carrier. The composition of the invention may be provided in any of the products defined herein.

In one embodiment, the composition for use according to the invention can be dried into powder and utilized in a portable sachet. For example, upon mixing such a powder with a suitable solvent such as water, a rinse may be created to remove plaque, proteins and other debris in the mouth.

In another embodiment, the composition for use according to the invention can be dried with abrasives such as silica, calcium carbonate or soft capsules that upon addition of small amount of water, creates a paste to brush away the plaque.

Formulations that increase the substantivity of ionic liquids onto a surface could be expected to increase the efficacy of biofilm, and hence plaque removal. For example, Tween 20 while also functioning as a surfactant, is also a wetting agent. Therefore, incorporation of such an agent could be expected to increase the wettability and spreading of a mouth rinse formulation according to the present invention, over the soft and hard tissue, increasing the formulation's propensity for plaque dissolution and removal.

### Methods of Use

The composition for use according to the present invention may be administered to or applied to a human or other animal subject. The composition may be suitable for administration or application to the oral cavity of a human or animal subject. Typically, the composition is for use in reducing or removing dental plaque. The reduction or removal of plaque may occur through an inhibition of biofilm (a plaque precursor) formation and/or degradation of microbial biofilm. Accordingly, the present invention further provides an oral composition comprising a therapeutically effective amount at least one ionic liquid for use in inhibiting biofilm formation and/or degrading biofilm in the oral cavity of a subject. Optionally, the composition is for use in teeth whitening.

The present invention further provides a composition as defined above for use in preventing or treating a disease condition of the oral cavity. Typically, the disease condition is caused by plaque. The disease condition may be selected from tooth decay, periodontal disease, gingivitis or xerostomia (dry mouth).

Accordingly, the present invention provides a composition as defined above for use as a medicament.

The present invention also provides a method of removing or reducing plaque from the oral cavity of a subject comprising administering a therapeutically effective amount of a composition comprising at least one ionic liquid to the subject. Preferably, the composition is an oral care composition as defined herein, and the composition is applied to the oral cavity.

In a preferred embodiment, the method is for treating or preventing a condition caused by plaque. Preferably, condition caused by plaque is selected from tooth decay, periodontal disease, gingivitis or xerostomia.

The present invention further provides a method of inhibiting biofilm formation and/or degrading biofilm in the oral cavity of a subject comprising administering to the subject an oral care composition comprising a therapeutically effective amount of an ionic liquid. Preferably, the oral care composition is as defined herein.

The present invention yet further provides a method of reducing the amount of bacteria in the oral cavity of a subject comprising administering to the subject an oral care composition comprising a therapeutically effective amount of an ionic liquid, wherein the oral care composition is selected from a mouth rinse, toothpaste, toothpowder, oral bead or strip, fluid-encased dental strip, irrigation fluid, plaque removal liquid, dental floss, hard candy, soft candy, lozenge, chewing gum, or lollipop. Preferably, the oral care composition is as defined herein.

The present invention still further provides a method of whitening teeth in a subject comprising administering a therapeutically effective amount of a composition comprising at least one ionic liquid to the subject. Preferably, the composition is an oral care composition as defined herein, and the composition is applied to the oral cavity. The oral care composition is preferably as defined herein.

Additionally, the present invention provides a use of an ionic liquid, in an oral care composition, for removing or reducing plaque in the oral cavity of a subject. The oral care composition is preferably as defined herein.

The present invention also provides a use of an ionic liquid, in an oral care composition, for whitening teeth in a subject. The oral care composition is preferably as defined herein.

The present invention further provides a use of an ionic liquid, in an oral care composition, for inhibiting biofilm formation, and/or degrading biofilm in the oral cavity of a subject. The oral care composition is preferably as defined herein.

The present invention still further provides a use of an ionic liquid, in an oral care composition, for reducing the amount of bacteria the oral cavity of a subject, wherein the oral care composition is selected from a mouth rinse, toothpaste, toothpowder, oral bead or strip, fluid-encased dental strip, irrigation fluid, plaque removal liquid, dental floss, hard candy, soft candy, lozenge, chewing gum, or lollipop. The oral care composition is preferably as defined herein.

The present inventors have unexpectedly found that compositions comprising an ionic liquid are highly effective in inhibiting the growth of bacteria, degrading biofilms, and dissolving dental plaque. They possess the unique ability to offer a deep but gentle cleaning, and promote removal of biofilm and plaque without the need for harsh abrasives or rigorous brushing. The compositions are further able to remove stains and whiten teeth, again without the need for harsh abrasives or rigorous brushing.

The invention is further illustrated in the following non-limiting examples.

### EXAMPLES

### Example 1

### Dissolution of Plaque

Dental plaque samples were obtained from subjects and treated with water to remove any water soluble actives. The samples were centrifuged at 13,000 g and the supernatant was collected and discarded. The remaining plaque that was insoluble in water was used to evaluate the plaque solubilizing potential of the ionic liquids.

Ionic liquids were added to the plaque sample, and the plaque sample was vortex mixed at room temperature. To ensure that the plaque was not simply suspended in the ionic liquid, the plaque containing vial with the ionic liquid was subsequently centrifuged at 13,000 g. Any undissolved plaque was visible as a pellet, and in this way, plaque dissolution could be visually monitored.

Plaque may form a suspension when mixed with water and that, on centrifugation, it is visible as an insoluble pellet. On addition of 1-ethyl-3-methylimidazolium bromide, the size of the plaque pellet is reduced, indicating that 1-ethyl-3-methylimidazolium bromide is effective in dissolving plaque. Similar results are obtained with choline acetate, choline salicylate and 1,2,4-trimethylpyrazolium methylsulfate.

### Example 2

### Protocol for In Vitro Biofilm Removal

A 3-day old biofilm provided a sufficiently robust biofilm to help differentiate the efficacy of different prototype formulations in inhibiting biofilm formation and/or degrading biofilm (biofilm removal).

The 3-day old biofilm was grown on a 24 well plate using the artificial mouth consortium of bacteria (A. naeslundii, S. oralis, V. parvula, L. casei, F. nucleatum) and S. mutans. To generate the biofilm, plates were inoculated with saliva overnight to form a pellicle. The consortium of bacteria (1 mL) at an OD of ˜0.2 was added to each well. The bacterial media (Tryptic Soy Broth with 6% sucrose) was changed after 48 hours. The 3-day old biofilm was treated with the prototype formulation (500 μL) for 15 minutes on a plate shaker at 300 rpm. After incubation, the supernatant was discarded. The optical density of each well was measured and the percentage reduction in optical density relative to the control (water) was calculated to determine the effect of the prototype formulation on biofilm removal.

As described herein, dental plaque is formed from a biofilm precursor. Therefore, the 3-day old biofilm serves as a good model to investigate the effects of test compounds on plaque removal.

### Example 3

### Impact of Chain Length on Biofilm Removal

Using the protocol of Example 2, the effects of ionic liquids on biofilm reduction were tested.

Water, untreated biofilm, commercially available mouthwash formulations with CPC (Cetyl pyridinium chloride) and a mouthwash containing five enzymes for combatting biofilm acted as negative controls for the experiment. Table 1 below is illustrative for different ionic liquids, formulated in the base prototype mouth rinse formation.

TABLE 1

| Ingredient Name | Percentage |
|---|---|
| Demineralized water | 38.63 |
| Propylene Glycol | 10.00 |
| Sorbitol non-browning/non-crys | 20 |
| NF-sol | |
| 99.0%-101.0% Vegetable | 20 |
| Glycerin | |
| Surfactant - Tween 20 | 1.125 |
| Flavor K91-6525 | 0.12 |
| CPC | 0.075 |
| Sucralose | 0.05 |
| Ionic Liquids | 8-10 |

The ionic liquids tested were as follows: 1-ethyl-3-methylimidazolium (EMIM) chloride (Cl), EMIM bromide (Br), EMIM ethyl sulfate, EMIM diethyl phosphate, EMIM acetate (OAc), EMIM tosylate, 1-butyl-3-methylimidazolium (BMIM) chloride (Cl), BMIM bromide (Br), BMIM methyl sulfate, BMIM octyl sulfate, BMIM acetate (OAc), 1-allyl-3-methylimidazolium (AMIM) chloride, 1-decyl-3-methylimidazolium (DMIM) chloride, 1,2,3-trimethylimidazolium methyl sulfate, 1,2,4-trimethylpyrazolium (MMMPZ) methyl sulfate, and tris(2-hydroxyethyl)methylammonium methylsulfate (MTEOA) methyl sulfate.

Table 2 represents the percentage removal of a 3-day old biofilm using 0.28M concentration of different ionic liquids, formulated in the base prototype mouth rinse formation of Table 1, as compared to control compositions water, mouthwash containing 0.075% CPC and mouthwash containing five enzyme to control biofilm, and untreated biofilm.

TABLE 2

| | Percent Removal of a 3-day |
|---|---|
| Sample Name | Oral Biofilm |
| Untreated | −1.3 |
| DI water | −3.7 |
| MW containing 0.075% | 13.4 |
| CPC | |
| MW containing five | 30.0 |
| enzymes | |
| EMIM Cl | 44.3 |
| BMIM Cl | 43.8 |
| AMIM Cl | 44.8 |
| DMIM Cl | 57.3 |
| EMIM Br | 48.6 |
| BMIM Br | 48.0 |
| BMIM MeSO4 | 53.9 |
| TMIM MeSO4 | 51.8 |
| MTEOA MeSO4 | 52.8 |
| MMMPz MeSO4 | 57.8 |
| EMIM Ethyl SO4 | 48.1 |
| BMIM Octyl SO4 | 58.6 |
| EMIM diethyl PO4 | 52.7 |
| BMIM OAc | 55.9 |
| EMIM OAc | 52.1 |
| EMIM Tosylate | 59.5 |

As seen in Table 2, each of the formulated ionic liquids were effective in removing oral biofilm by at least 40% or more. In particular, ionic compounds comprising an imidazolium cation, and an anion selected from acetate, halide, alkyl phosphate, alkyl sulfate, and tosylate, are significantly more effective than the commercially available or control compositions in reducing biofilm formation. These compounds remove biofilm by an amount that is 2 to 6 times more than the amount removed by the commercially available mouth rinses.

Additionally, it can be seen from Table 2 that biofilm removal increases as the length of the side chain on the imidazolium cation (at the 1-position) is increased. In particular, DMIM chloride is significantly more effective in removing biofilm than EMIM chloride and BMIM chloride.

Furthermore, it can be seen from Table 2 that when the counterion is changed from chloride to bromide for EMIM and BMIM, there is an increase in biofilm removal by at least ˜4%.

Thus, it may be concluded that ionic liquids having a longer chain in their core cation moiety and having a larger halide ion such as a bromide ion, enables greater biofilm removal.

Table 2 also reveal that in general, methyl sulfate anions perform better than the halide ions. Additionally, when the alkyl chain of the sulfate ion is increased, (for example, biofilm removal for BMIM methyl sulfate and BMIM octyl sulfate may be compared), biofilm removal is markedly enhanced.

Therefore, in conclusion, compositions comprising ionic liquids, in particular, wherein the ionic liquids comprise an imidazolium ion, are very effective in removing 3-day old biofilm, which serves as a model for removing plaque.

### Example 4

### Impact of Anion on Biofilm Removal

Using the protocol of Example 2, the effects of varying the anion type in EMIM- and BMIM-based ionic liquids, on removal of a 3-day old biofilm, were investigated.

Table 3 illustrates the percentage removal of a 3-day old biofilm using 0.28M concentration of different EMIM-based ionic liquids having different anions, formulated in the base prototype mouth rinse formation of Table 1.

TABLE 3
EMTM Cationic Series

| | Percent Biofilm |
|---|---|
| Anion | Removed |
| Cl | 44.3 |
| Ethylsulfate | 48.1 |
| Br | 48.6 |
| Acetate | 52.1 |
| Diethyl | 52.7 |
| phosphate | |
| Tosylate | 59.5 |

Table 4 illustrates the percentage removal of a 3-day old biofilm using 0.28M concentration of different BMIM-based ionic liquids having different anions, formulated in the base prototype mouth rinse formation of Table 1.

TABLE 4
BMIM Cationic Series

| | Percent Biofilm |
|---|---|
| Anion | Removal |
| Cl | 43.8 |
| Br | 48.0 |
| Methylsulfate | 53.9 |
| Acetate | 55.8 |
| Octylsulfate | 58.6 |

Table 3 illustrates that the anions diethyl phosphate, acetate and tosylate are most effective in removing biofilm. Efficacy of biofilm removal appears to increase with negatively resonance stabilized species such as acetate and tosylate.

Table 4 illustrates that acetate and octylsulfate anions are more effective in removing biofilm than the halide ions.

Table 5 illustrates a direct comparison between EMIM and BMIM, with different anions, in removing biofilm.

TABLE 5

Percent Biofilm Removed

| | EMIM | BMIM |
|---|---|---|
| Chloride | 44.3 | 43.8 |
| Bromide | 48.6 | 48.0 |
| Acetate | 52.1 | 55.8 |

It can clearly be seen that acetate anions are most effective in removing biofilm, followed by bromide ions and chloride ions, respectively.

### Example 5

### Biofilm Removal by Other Imidazolium Compounds

Using the protocol of Example 2, the effects of 1-allyl-3-methylimidazolium (AMIM) chloride, 1,2,3-trimethylimidazolium (TMIM) methyl sulfate, and 1-decyl-3-methylimidazolium (DMIM) chloride on removal of a 3-day old biofilm, were compared. The results are illustrated in Table 6.

TABLE 6

| Other Ionic | Percent Biofilm |
|---|---|
| Liquids | Removed |
| AMIM Chloride | 44.8 |
| TMIM | 51.8 |
| Methylsulfate | |
| MTEOA | 52.8 |
| Methylsulfate | |
| DMIM Chloride | 57.3 |
| MMMPz | 57.9 |
| Methylsulfate | |

It can clearly be seen from Table 6 that DMIM chloride is most effective in removing biofilm. It can also be concluded that imidazolium cations with unsaturated side chains (e.g. AMIM chloride) are effective in removing biofilm.

### Example 6

### Teeth Whitening Effect of Imidazolium-Based Ionic Liquids

Mouthwash formulations of 1-ethyl-3-methylimidazolium (EMIM) with the anions tosylate, bromide, chloride and ethylsulfate, were evaluated for in-vitro removal of tooth stains.

The method of determining the effects of imidazolium-based ionic liquids on teeth whitening was carried out as follows:

The initial L*a*b* measurements of dried artificially stained human teeth were captured. The teeth were then soaked in 1 ml of sample solution for one hour, and sample was replenished at 30 minutes. After treatment, teeth were soaked in DI water for approximately 10 minutes. The teeth were left to dry at least overnight, and final L*a*b* measurements were then taken. (L*a*b* refers to stain score in accordance with the Commission International de L'Eclairage Laboratory (CIELAB) color scale. L* (lightness-darkness scale), a* (red-green chroma) and b* (yellow-blue chroma)).

The whitening efficacy was determined as follows:

ΔW*=W*final−W*initial

where W*=(a*2+b*2+(L*−100)2)½

All the EMIM-based compounds were evaluated at 1M concentration in a prototype mouth wash formulation as illustrated in Table 7.

TABLE 7
Prototype mouth wash formulation containing
EMIM-based ionic liquids at 1M concentration.
Prototype Whitening MW Base

| Ingredient Name | Weight % |
|---|---|
| Glycerin | 0.000 |
| Propylene Glycol | 2.500 |
| Sorbitol | 10.000 |
| Surfactant-Tween 20 | 1.500 |
| Ionic Liquid Hole (1M) | 30.000 |
| Sodium Benzoate | 0.110 |
| CPC | 0.075 |
| Sucralose | 0.050 |
| Citrate Buffer | 10.000 |
| Flavor (K91-5916) | 0.150 |
| Water | 45.615 |
| Total | 100.000 |

Change in tooth whiteness effected by various EMIM-based liquids were used as peroxide-containing controls for the experiment. It can be seen that the tooth whitening capacities of the imidazolium-based ionic liquids decrease in the order: EMIM tosylate, EMIM Br, and EMIM Cl/EMIM ethyl sulfate. (EMIM Cl and EMIM ethyl sulfate produced similar whitening effects). Changes in teeth whiteness (W), shade (E) and lightness (L) were also assessed. The results are illustrated in Table 8.

TABLE 8

| Sample Name | ΔW | ΔE | ΔL |
|---|---|---|---|
| EMIM EtSO4 | −2.99 | 4.41 | 3.40 |
| EMIM Br | −7.98 | 9.75 | 8.86 |
| EMIM Cl | −3.40 | 4.88 | 3.86 |
| EMIM Tos | −13.90 | 15.53 | 15.03 |
| Stain guard | −3.73 | 4.05 | 3.96 |
| formulation | | | |
| Hydrogen peroxide | −17.55 | 22.20 | 20.68 |
| containing MW | | | |

Again, EMIM-tosylate, a non-peroxide, provided the most significant tooth whitening benefit through dissolution and removal of surface tooth stains.

### Example 7

### Teeth Whitening Effect of Pyrazolium-Based Ionic Liquids

Table 9 illustrates a general mouth rinse formulation comprising 1,2,4-trimethylpyrazolium methylsulfate.

TABLE 9

| | Level Range |
|---|---|
| Ingredient Name | (wt %) |
| Glycerin | 0-20 |
| Propylene Glycol | 0-10 |
| Sorbitol | 0-20 |
| 1,2,4-Trimethylpyrazolium methylsulfate | 5-20 |
| Na Benzoate | 0-0.11 |
| CPC | 0-0.075 |
| Surfactant* | 1.5 |
| Sucralose | 0.05 |
| Citrate Buffer | 10 |
| Flavor | 0.15 |
| Water | q.s |

*Polysorbate 20, Polysorbate 80, Pluronic F-108, Pluronic F-127, PEG 40

Three exemplary compositions according to the general formulation of Table 9 were tested in order to determine their whitening efficacy. The determination of the whitening efficacy was conducted as follows: After brushing and rinsing, artificially stained human teeth were allowed to dry and the initial L*a*b* readings recorded. The teeth were then soaked for 28 treatments, at 2 minutes per treatment, in 1 mL of the prototype whitening mouth rinse. The noted treatment time is typically used to equate 2 weeks' worth of consumer usage when brushing. The teeth were rinsed with DI water after every treatment. The whitening solutions were replenished every 7^{th} treatment. After 28 treatments, the teeth were rinsed with distilled water and allowed to dry. The final L*a*b* readings were then recorded. Whitening values were calculated using Equation 1 where ΔW (whitening) was used to quantify the whitening efficacy of each formula. Equation 1 (ΔW*=W*_{final} W*ᵢₙᵢₜᵢₐₗ and W*=a(*2+b*²+(L*−100)²)^{1/2})

The results of the whitening analysis are provided in Table 10. The more negative the change in Whiteness, ΔW, the whiter the appearance of teeth. The greater and more positive the ΔL value, the lighter the colour of the teeth. The compositions were compared to hydrogen peroxide containing whitening formulation and a formulation with stain guard technology to prevent stains from attaching to the surface of the teeth.

TABLE 10

| | Ingredient Level | | | | |
|---|---|---|---|---|---|
| | | | | Hydrogen | Stain |
| | | | | peroxide | guard |
| | | | | formu- | formu- |
| Ingredient Name | A | B | C | lation | lation |
| Glycerin | 0 | 0 | 10 | | |
| Propylene Glycol | 0 | 2.5 | 7 | | |
| Sorbitol | 20 | 10 | 2 | | |
| 1,2,4- | 20 | 20 | 20 | | |
| Trimethylpyrazolium | | | | | |
| methylsulfate | | | | | |
| Na Benzoate | 0.11 | 0.11 | 0.11 | | |
| CPC | 0.075 | 0.075 | 0.075 | | |
| Surfactant | Tween | Tween | Tween | | |
| | 20 | 20 | 20 | | |
| pH | 4 | 5 | 5 | | |
| ΔW | −23.54 | −25.29 | −21.05 | −19.22 | 1.68 |
| ΔL | 25.17 | 26.48 | 22.50 | 17.34 | −2.5 |

Tween 20 (polyoxyethylene sorbitan monolaurate)

As can be seen in Table 10, compositions A, B and C comprising 1,2,4-Trimethylpyrazolium methylsulfate provided greater whitening efficacy than the commercially available control compositions.

### Example 8

### In Vitro Removal of Biofilm

General Procedures Used to Prepare Biofilm

Saliva Preparation: Saliva was collected from the analyst who is a healthy adult with no history of antibiotic use in the previous week and who does not require routine medications. The analyst used a commercially available fluoride dentifrice (Colgate Pro Clinical) and a soft bristled toothbrush (Colgate 360) for routine oral hygiene for 3 days prior to the start of saliva collection. Saliva was collected in the morning, at least two hours after a meal and was centrifuged at 10,000 rpm for 20 minutes. The supernatant was decanted into a Petri dish and placed under UV for 45 minutes.

Bacteria Preparation: Bacteria were taken from the artificial mouth consortium (containing Actinomyces naeslundii, Streptococcus oralis, Veillonella parvula, Lactobacillus casei, and Fusibacterium nucleatum) and combined with Streptococcus mutans which had been grown separately in a ratio of 2:1. Bacteria were grown in tryptic soy broth (TSB) media with 6% sucrose. The resulting bacterial culture was diluted to the required volume, prior to diluting to an optical density of 0.2.

Plate Preparation: 500 μL of UV-treated saliva were added to each well of a 24-well plate. Plates were inoculated overnight in 37° C. After inoculation, excess saliva was discarded and 1 ml of bacteria was added to each well. The bacteria were allowed to attach and develop a biofilm on the bottom of each well over 2 to 4 days, changing the media every 48 hours. During media change, the old media was discarded and 1 ml of fresh media was added.

Treatment: 500 μL of ionic liquid formulation was added to each well and incubated for 4-30 minutes on a plate shaker at 300 rpm. After incubation, the supernatant was discarded. No rinsing was carried out. The optical density of each well was measured at 610 nm, and the percentage reduction in biofilm reduction relative to the control (water) was calculated.

Second Treatment: 500 μL of ionic liquid formulation was added to each well and incubated for 4-30 minutes on a plate shaker at 300 rpm. After discarding the supernatant, the optical density was measured and the percentage reduction in biofilm formation relative to the control (water) was calculated.

Prototype Mouthrinse Formulations

A statistical design-of-experiments (DOE) approach was used to develop prototype mouth rinse formulations for biofilm removal. An 8 factor design was used. Colgate Plax Re-launch mouth rinse was used a base formulation to build upon. The impact of five different surfactants in varying amounts (0.75-1.5%) was determined, in addition to the impact of the ionic liquids choline salicylate and tris(2-hydroxyethyl)methylammonium methylsulfate (Tris-HMAM), used either alone, or in combination, in the formulations. The influence of the anti-bacterial agent cetyl pyridinium chloride (CPC) on biofilm removal was also evaluated. The sweetener sucralose and the flavor were at kept constant concentration in all the formulations. Table 11 illustrates the general formulation on the basis of which the prototype formulations were designed.

TABLE 11

| Ingredient | Amount (%) |
|---|---|
| Glycerin | 0-20 |
| Propylene Glycol | 0-10 |
| Sorbitol | 0-20 |
| Surfactant (Tween 80, Tween 20, PEG 40, Pluronic F108, | 0.75-1.5 |
| or Pluronic F127) | |
| Choline Salicylate and/or | 0-20 |
| Tris (2-hydroxyethyl)methyl ammonium methylsulfate | |
| Sucralose | 0.05 |
| Flavor | 0.12 |
| CPC | 0-0.075 |
| Water | balance |

74 prototype formulations were developed. There are shown below in Table 12 (see next page). Table 12 further illustrates the percentage biofilm reduction after one 15-minute treatment of a 3-day-old biofilm with the formulation, and the viscosity for each formulation. In Table 12, PG is propylene glycol; CPC is cetyl pyridinium chloride; and TRIS is tris(2-hydroxyethyl)methylammonium methylsulfate. All amounts are given in wt %.

A—Standard Formulation number

### B—Glycerin

C—Polyethylene glycol

### D—Sorbitol

E—CPC (cetyl pyridinium chloride)
F—Surfactant level
G—Choline salicylate
H—TRIS (tris(2-hydroxyethyl)methylammonium methylsulfate)
I—Surfactant type
J—% Biofilm reduction

### K—Viscosity (cP)

TABLE 12

| A | B | C | D | E | F | G | H | I | J | K |
|---|---|---|---|---|---|---|---|---|---|---|
| 32 | 20 | 5 | 20 | 0 | 0.75 | 20 | 20 | F108 | 80 | 47 |
| 21 | 20 | 0 | 0 | 0 | 1.5 | 20 | 20 | Tween80 | 80 | 8 |
| 7 | 0 | 10 | 20 | 0 | 0.75 | 20 | 20 | Tween20 | 79 | 11 |
| 8 | 20 | 10 | 0 | 0 | 1.5 | 20 | 20 | PEG40 | 78 | 14 |
| 19 | 20 | 10 | 0 | 0.075 | 1.5 | 20 | 20 | Tween20 | 78 | 13 |
| 50 | 20 | 5 | 20 | 0.075 | 0.75 | 20 | 0 | Tween80 | 77 | 11 |
| 9 | 0 | 10 | 20 | 0.075 | 0.75 | 20 | 20 | F108 | 77 | 13 |
| 2 | 0 | 10 | 20 | 0 | 0.75 | 20 | 20 | PEG40 | 77 | 13 |
| 29 | 20 | 10 | 10 | 0 | 0.75 | 20 | 20 | Tween20 | 76 | 27 |
| 1 | 20 | 10 | 20 | 0.075 | 1.5 | 20 | 20 | Tween80 | 76 | 78 |
| 31 | 10 | 10 | 0 | 0 | 1.5 | 20 | 20 | F127 | 76 | 10 |
| 10 | 0 | 10 | 20 | 0 | 1.5 | 20 | 20 | F108 | 75 | 16 |
| 18 | 0 | 10 | 20 | 0.075 | 1.5 | 20 | 20 | F127 | 74 | 18 |
| 24 | 20 | 10 | 20 | 0.075 | 0.75 | 20 | 20 | PEG40 | 74 | 70 |
| 15 | 20 | 10 | 20 | 0 | 1.5 | 0 | 20 | F127 | 74 | 17 |
| 60 | 20 | 10 | 20 | 0.075 | 1.125 | 20 | 0 | F127 | 74 | 19 |
| 51 | 0 | 5 | 0 | 0.075 | 0.75 | 20 | 0 | F127 | 73 | 2 |
| 42 | 20 | 10 | 20 | 0.0375 | 1.5 | 0 | 20 | PEG40 | 73 | 15 |
| 13 | 20 | 0 | 20 | 0.075 | 1.5 | 20 | 20 | F127 | 73 | 35 |
| 39 | 20 | 5 | 0 | 0.075 | 0.75 | 20 | 20 | Tween80 | 73 | 10 |
| 20 | 20 | 0 | 20 | 0.075 | 1.5 | 20 | 20 | F108 | 73 | 42 |
| 40 | 20 | 0 | 20 | 0.0375 | 1.5 | 20 | 0 | Tween20 | 71 | 9 |
| 12 | 20 | 0 | 20 | 0 | 1.5 | 0 | 20 | Tween20 | 70 | 8 |
| 16 | 20 | 10 | 0 | 0.075 | 1.5 | 20 | 20 | F108 | 70 | 20 |
| 56 | 20 | 0 | 20 | 0.075 | 0.75 | 10 | 20 | Tween20 | 70 | 13 |
| 34 | 10 | 10 | 0 | 0 | 1.5 | 20 | 20 | Tween20 | 70 | 7 |
| 49 | 10 | 10 | 20 | 0 | 0.75 | 20 | 0 | Tween80 | 69 | 8 |
| 52 | 0 | 0 | 20 | 0 | 1.125 | 20 | 0 | F127 | 69 | 3 |
| 61 | 20 | 0 | 0 | 0.0375 | 0.75 | 20 | 20 | Tween20 | 69 | 7 |
| 33 | 0 | 0 | 20 | 0 | 1.5 | 10 | 20 | Tween80 | 69 | 4 |
| 22 | 0 | 0 | 10 | 0.075 | 0.75 | 20 | 20 | F127 | 68 | 4 |
| 57 | 0 | 10 | 20 | 0.075 | 0.75 | 10 | 20 | Tween80 | 68 | 7 |
| 35 | 0 | 5 | 20 | 0 | 1.5 | 20 | 0 | PEG40 | 68 | 4 |
| 62 | 20 | 10 | 0 | 0 | 0.75 | 10 | 0 | F108 | 67 | 4 |
| 27 | 0 | 0 | 10 | 0.075 | 1.5 | 20 | 20 | PEG40 | 67 | 4 |
| 30 | 20 | 0 | 20 | 0.0375 | 0.75 | 0 | 20 | Tween80 | 67 | 8 |
| 28 | 0 | 10 | 20 | 0 | 1.125 | 0 | 20 | F108 | 67 | 5 |
| 5 | 20 | 10 | 0 | 0 | 1.5 | 20 | 0 | Tween20 | 67 | 6 |
| 38 | 0 | 0 | 10 | 0.075 | 1.5 | 20 | 20 | Tween80 | 65 | 5 |
| 36 | 0 | 10 | 10 | 0.075 | 1.5 | 20 | 0 | Tween80 | 64 | 3 |
| 3 | 20 | 0 | 20 | 0 | 1.5 | 0 | 20 | F108 | 63 | 11 |
| 4 | 20 | 10 | 0 | 0.075 | 0.75 | 0 | 20 | F127 | 63 | 5 |
| 70 | 20 | 10 | 0 | 0.075 | 0.75 | 20 | 0 | PEG40 | 62 | 5 |
| 64 | 20 | 0 | 20 | 0 | 0.75 | 10 | 0 | PEG40 | 61 | 5 |
| 26 | 0 | 0 | 10 | 0.075 | 1.5 | 20 | 20 | F108 | 59 | 6 |
| 6 | 0 | 10 | 0 | 0 | 1.5 | 0 | 20 | Tween80 | 57 | 2 |
| 59 | 0 | 10 | 20 | 0 | 1.5 | 10 | 0 | Tween20 | 56 | 1 |
| 63 | 0 | 5 | 20 | 0.075 | 1.5 | 0 | 20 | Tween20 | 55 | 4 |
| 23 | 0 | 5 | 20 | 0 | 1.5 | 20 | 0 | F108 | 54 | 5 |
| 41 | 20 | 10 | 20 | 0 | 1.5 | 0 | 0 | Tween80 | 54 | 6 |
| 53 | 0 | 0 | 20 | 0 | 1.5 | 0 | 10 | F127 | 53 | 3 |
| 14 | 0 | 0 | 0 | 0.075 | 0.75 | 0 | 20 | PEG40 | 52 | 2 |
| 65 | 0 | 0 | 0 | 0.075 | 1.125 | 0 | 20 | F127 | 50 | 2 |
| 72 | 0 | 10 | 0 | 0 | 1.5 | 0 | 20 | Tween80 | 50 | 3 |
| 67 | 0 | 5 | 0 | 0 | 0.75 | 20 | 0 | PEG40 | 50 | 2 |
| 47 | 20 | 0 | 20 | 0.075 | 0.75 | 0 | 0 | F108 | 49 | 4 |
| 66 | 0 | 0 | 0 | 0 | 0.75 | 0 | 10 | Tween80 | 49 | 1 |
| 46 | 10 | 10 | 20 | 0.075 | 0.75 | 0 | 0 | Tween20 | 49 | 4 |
| 37 | 10 | 0 | 0 | 0.075 | 1.5 | 0 | 20 | Tween80 | 47 | 2 |
| 73 | 20 | 10 | 0 | 0 | 1.5 | 20 | 0 | Tween20 | 43 | 6 |
| 71 | 20 | 0 | 0 | 0 | 0.75 | 0 | 20 | F127 | 43 | 3 |
| 44 | 0 | 10 | 20 | 0 | 0.75 | 0 | 0 | F127 | 43 | 3 |
| 54 | 10 | 5 | 10 | 0.0375 | 1.125 | 0 | 10 | Tween20 | 42 | 3 |
| 74 | 20 | 0 | 20 | 0.075 | 1.5 | 0 | 0 | PEG40 | 41 | 4 |
| 69 | 20 | 0 | 0 | 0.075 | 1.125 | 20 | 0 | F108 | 41 | 4 |
| 17 | 20 | 0 | 20 | 0.075 | 1.5 | 0 | 0 | PEG40 | 40 | 4 |
| 43 | 0 | 0 | 0 | 0 | 1.5 | 0 | 0 | Tween20 | 31 | 1 |
| 11 | 0 | 10 | 0 | 0.075 | 1.5 | 0 | 0 | PEG40 | 31 | 2 |
| 45 | 20 | 0 | 0 | 0 | 1.125 | 0 | 0 | PEG40 | 29 | 2 |
| 55 | 0 | 10 | 0 | 0.0375 | 1.5 | 0 | 0 | F108 | 29 | 2 |
| 48 | 0 | 0 | 0 | 0 | 0.75 | 20 | 0 | F108 | 26 | 2 |
| 25 | 20 | 0 | 10 | 0.075 | 1.5 | 0 | 0 | F127 | 20 | 3 |
| 58 | 0 | 0 | 20 | 0.0375 | 0.75 | 0 | 0 | PEG40 | 15 | 2 |
| 68 | 0 | 10 | 10 | 0.075 | 0.75 | 0 | 0 | F108 | 12 | 2 |

F108 (Synperonic ® F-108 (poly(ethylene glycol)-block-poly(propylene glycol)-block- poly(ethylene glycol)); F127 (Pluronic ® F-127 (polyoxypropylenepolyoxyethylene block copolymer); PEG40 (ethylene oxide); Tween 20 (polyoxyethylene sorbitan monolaurate); Tween 80 (polyoxyethylene sorbitan monooleate)

As can be seen from the results in Table 4, twenty-six (26) of the prototype formulations provided about >70% biofilm removal. Generally, formulations containing quaternary ammonium compounds meeting the >70% biofilm removal can be predicted if the formulation met the following criteria:

(i) at least one of glycerin or polyethylene glycol is present;
(ii) no more than two of glycerin (B), polyethylene glycol (C), sorbitol (D), CPC (cetyl pyridinium chloride) (E), surfactant (F), choline salicylate (G) and (tris(2-hydroxyethyl)methylammonium methylsulfate)(H) is present, unless the wt % sum of B+C+D is less than the wt % sum of F+G; and
(iii) a biofilm factor (BF) of at least 50, wherein the BF=(wt % of B+C+D+G+H)×(wt % of E+F).

As can be seen from the results in Table 12, twenty-four of the prototype formulations provided at least 70% biofilm removal. In contrast, commercial products exhibit less than 30% biofilm removal.

The above results strongly suggest that several of the formulations identified through the design-of-experiments approach and may provide clinical efficacy to disrupt, dissolve and remove plaque in vivo.

### Example 9

### Efficacy of Dissolution of Morning Salivary Sediment

Several of the prototype formulations of the present invention provide significant dissolution of early morning salivary sediment. The salivary sediment was collected without evening or early morning brushing, and the salivary supernatant was removed by decanting. Such salivary sediment contains appreciable amounts of dental plaque and left over food debris that is very difficult to dissolve and remove from the oral cavity without brushing with dentifrice. By way of illustration, 1-10 mL, for example 5 mL of the prototype formulations or the commercial products (a CPC containing MW, Listerine and Crest Pro-Health) was added to the salivary sediment in a container and the effects on salivary sediment dissolution were noted after 30 seconds to 60 seconds of shaking or vortexing the container.

Commercial mouth rinses such a CPC containing MW, Listerine (J&J) and Crest Pro-Health (P&G) showed no early morning salivary sediment dissolution. However, it was found that formulations of the present invention helped to dissolve salivary sediment.

In general, there was a positive correlation between those formulations of the present invention that exhibited 70% biofilm removal in-vitro and salivary sediment dissolution.

### Example 10

### Factors and Ingredients that Affect Biofilm Removal

Concentration of Ionic Liquids

It was found that an increase in the amount of ionic liquid used in the formulation results in greater biofilm removal. The percentage biofilm removal provided by each ionic liquid formulation is similar ca. 60%. When the two ionic liquids choline salicylate and tris(2-hydroxyethyl)methylammonium methylsulfate are used together in a formulation, the percent biofilm efficacy is increased further to >70%.

Impact of Different Ingredients in the Formulation

Testing to determine the impact of different ingredients on biofilm removal is shows that the identity of the surfactant, the percentage of surfactant used and the amount of anti-bacterial agent cetyl pyridinium chloride (0 to 0.075%) do not impact biofilm removal.

As seen on moving from the upper left panel to the upper right panel in FIG. 11, addition of glycerin, sorbitol and/or propylene glycol to the formulation increases percentage biofilm removal.

Taking all these results together, a few significant formulation design criteria emerge for the effective removal of a robust 3-day old in-vitro oral biofilm. These are summarized below:
Increase in choline salicylate or tris(2-hydroxyethyl)methylammonium methylsulfate provides significant increase in biofilm removal;
Increase in humectants increases biofilm removal: sorbitol>glycerin≈propylene glycol;
Surfactant and cetyl pyridinium chloride (CPC) do not affect biofilm removal.

Table 13 illustrates the effects of choline salicylate and tris(2-hydroxyethyl)methylammonium methylsulfate alone, or in combination, on biofilm removal, in the presence and absence of sorbitol at 20%.

As seen in Table 13, 20% sorbitol in the formulations provides ˜5% increase in biofilm removal. (These formulations also contain 10% glycerin and 5% propylene glycol 5%).

TABLE 13

| | TRIS-based | | Percent biofilm |
|---|---|---|---|
| Choline | ionic | Percent biofilm | removal if |
| salicylate | liquid | removal | sorbitol at 20% |
| 0 | 0 | 35 | 40 |
| 0 | 10 | 47 | 52 |
| 0 | 20 | 60 | 65 |
| 10 | 0 | 48 | 53 |
| 20 | 0 | 61 | 66 |
| 10 | 10 | 57 | 62 |
| 20 | 20 | 72 | 77 |
| 10 | 20 | 66 | 71 |
| 20 | 10 | 60 | 65 |

TRIS = tris (2-hydroxyethyl) methylammonium methylsulfate

Table 14 (below) shows the optimal combination of ingredients for effective removal of a 3-day biofilm, as predicted utilizing the D-Optimal Design module in the Design-Expert® Software program. As discussed above, the surfactant type (of the five chosen) and level (tested range is 0.75-1.5%) do not significantly affect biofilm dissolution. Flavor and sucralose also do not significantly affect biofilm dissolution when used at 0.05% and 0.12% respectively. The presence (0.075%) or absence of cetyl pyridinium chloride (CPC) further does not affect biofilm dissolution.

TABLE 14

| Ingredient | Amount (%) |
|---|---|
| Glycerin | 20 |
| Propylene glycol | 10 |
| Sorbitol | 20 |
| Surfactant (Tween 80, Tween 20, PEG 40, | 1.125* |
| Pluronic F108, Pluronic F127) | |
| Choline salicylate | 20 |
| Tris (2-hydroxyethyl) methylammonium | 20 |
| methylsulfate | |
| Sucralose | 0.05 |
| Flavour | 0.12 |
| CPC | 0.075 |
| Water | balance |

*surfactant type and level (0.75-1.5). CPC, flavour, sucralose at constant value
Predicted 3-day biofilm removal: 83.66%
Stdev: 4.56, pH = 7.26

### Example 11

### Enhanced Mouthrinse Compositions and their Efficacy for Biofilm Removal

From the design-of-experiments results above and the design criteria, mouthrinse formulations containing tris(2-hydroxyethyl)-methylammonium methylsulfate at different percent concentrations were developed.

Optimized Mouth Rinse Formulations

Different concentrations of the ionic liquid, tris(2-hydroxyethyl)methylammonium methylsulfate (Tris-HMAM), in low and high-humectant mouth rinse formulations were assessed for their ability to remove a 3-day old multispecies oral biofilm and compared to the controls DI water, Crest Pro-Health (0.075% CPC) and Biotene PBF (five enzyme containing formulation).

Low-humectant mouthrinse formulations may contain 0-15% glycerin, 0-7% propylene glycol and 0-20% sorbitol. High-humectant mouthrinse formulations may contain 15-40% glycerin, 10-40% propylene glycol and 20-40% sorbitol. Table 15 illustrates representative formulations.

TABLE 15

| | LOW humectant | HIGH humectant |
|---|---|---|
| Ingredient | amount % | amount % |
| Glycerin | 15 | 20 |
| Propylene glycol | 7 | 10 |
| Sorbitol | 10.5 | 20 |
| CPC | 0.075 | 0.075 |
| Tween 20 | 1.125 | 1.125 |
| Tris-HMAM | 0-40 | 0-40 |
| Sucralose | 0.05 | 0.05 |
| Flavour | 0.12 | 0.12 |
| water | balance | balance |

It was found that aging of the above formulations provided no stability or cosmetic issues. Other humectants may be used in place of those above.

Table 16 (below) illustrates the percentage removal of a 3-day old biofilm with different concentrations of tris(2-hydroxyethyl)methylammonium methylsulfate (Tris-HMAM) in low and high humectant mouth rinse formulation. As can be seen in Table 16, both low and high humectant formulations are effective in removing a 3-day-old multispecies oral biofilm:

TABLE 16

| Conc Tris-HMAM | 0% | 1% | 2.5% | 5% | 10% | 20% | 40% |
|---|---|---|---|---|---|---|---|
| % removal in Low | 12.4 | 25.6 | 28.2 | 30.9 | 46.8 | 57.3 | 67.1 |
| humectant | | | | | | | |
| mouthwash | | | | | | | |
| % removal in High | 37.3 | 39.0 | 50.5 | 53.9 | 57.7 | 71.1 | 73.2 |
| humectant | | | | | | | |
| mouthwash | | | | | | | |

It was determined that the controls deionised water and a commercial mouthwash containing 0.075% CPC removed less than 1% biofilm. A commercial mouthwash containing five enzymes in its formulations, removed 31.7% biofilm.

The results of Table 16 shows that (1) the high humectant formulation removes ca. three times as much biofilm than the low humectant formulation; (2) the addition of tris(2-hydroxyethyl)methylammonium methylsulfate to either low or high humectant mouth rinse formulation enhances biofilm removal; and (3) high humectant base mouthwash alone removes more biofilm than the commercially available five enzyme mouthwash formulation) and CPC containing mouth rinses.

Varying the type, amount and the ratios of the humectants to each other can be expected to further increase the efficiency and speed of biofilm removal. Similar behaviour can be expected of other quaternary based compounds containing an alkyl hydroxyl appendage or other H-bonding or donating end group.

### Example 12

### Antibacterial Activity of Imidazolium-Based Ionic Liquids

The effects of 1-decyl-3-methylimidazolium (DMIM) chloride on bacterial growth were investigated in vitro. DMIM chloride was incorporated into a prototype mouth rinse as illustrated in Table 17. Growth inhibition of A. viscosus was assessed when using DMIM chloride in an amount of 0.01 wt %, 0.1 wt % and 1 wt % in the prototype mouth rinse. The growth inhibition was measured as a change in optical density over time.

TABLE 17

| | Wt % Plaque dis- |
|---|---|
| Ingredient Name | solving formula |
| Demineralized water | Balance |
| Propylene Glycol | 4.00 |
| Sorbitol non-browning/non-crys | 7.00 |
| NF-sol | |
| 99.0%-101.0% Vegetable | 7.00 |
| Glycerin | |
| Flavor K91-6525 | 0.15 |
| Polyoxyl 40 hydrated castor oilNF | 1.00 |
| Sucralose | 0.001 |
| 1-decyl-imidazolium chloride | 0.3-10% |

1-decyl-3-methylimidazolium chloride formulated at 1 wt % concentration provides greater growth inhibition of A. viscosus than a 0.075 wt % CPC mouthwash and growth inhibition of A. viscosus is maintained even when 1-decyl-3-methylimidazolium chloride is formulated at 0.312 wt % concentration.

### Example 13

### Short Interval Kill Test (SIKT)

SIKT determines the kill effect of a test article at a predetermined exposure time. Briefly, a culture of A. viscosus was incubated with 1-decyl-3-methylimidazolium, formulated in the prototype mouth rinse according to Table 9. The reaction was neutralized after 30 seconds by adding a neutralizing broth. The reaction mixture was further diluted and plated on MCA (Microbial Count Agar) plates for viable bacterial count.

When 1-decyl-3-methylimidazolium chloride is formulated in the prototype mouth rinse according to Table 9 at 0.3, 0.6 and 1 wt % concentration, it is able to kill A. viscosus within 30 seconds.

### Example 14

### Minimum Inhibitory Concentration of 1-decyl-3-methylimidazolium Chloride

In order to determine the minimum concentration of 1-decyl-3-methylimidazolium chloride required to inhibit the growth of A. viscosus, an A. viscosus culture was incubated for 24 hours with varying concentrations of 1-decyl-3-methylimidazolium chloride, and bacterial growth inhibition was measured by taking optical density readings at 610 nm. Triclosan was used as a positive control for the experiment, and media alone was used as a negative control for the experiment.

The minimum inhibitory concentration of 1-decyl-3-methylimidazolium chloride required to inhibit the growth of A. viscosus is 125 ppm.

### Example 15

### Formulations of 1-decyl-3-methylimidazolium Chloride in Oral Care Delivery Vehicles

Table 18 shows a typical formulation of 1-decyl-3-methylimidazolium chloride in a bead formulation.

TABLE 18

| | Wt % Plaque dis- |
|---|---|
| Ingredient Name | solving WISP formula |
| Propylene glycol | Balance |
| Castor Oil | 43.5 |
| Flavor | 15 |
| WS-3 -Cooling sensate | 1.5 |
| 10% Sucralose sln | 5 |
| Ionic Liquid | 0.3-5 |

Table 19 shows a typical formulation of 1-decyl-3-methylimidazolium chloride in a wick delivery device or in an interdental wicking brush.

TABLE 19

| | Wt % Plaque dis- |
|---|---|
| Ingredient Name | solving formula |
| Glycerin | 15 |
| Propylene glycol | 30 |
| Flavor (89-186) | 8 |
| WS3-Cooling sensate | 3 |
| 10% Sucralose soln | 5 |
| Ionic Liquid Hole | 0.3-10 |
| Water pH adjusted if necessary | Balance |

### Example 16

### Antibacterial Efficacy of Choline Salicylate

The antibacterial effects of various ionic liquids were determined according to the protocol described in Example 12. Table 20 (below) illustrates that, when formulated at 10% concentration in a mouth rinse, choline salicylate provides a greater growth inhibition of A. viscosus than control compositions containing cetyl pyridinium chloride.

TABLE 20

| Ionic Liquids - Sample ID | 0 h | 4 h | 22 h | 24 h |
|---|---|---|---|---|
| A: 1,2,4-trimethyl-pyrazolium | 0.1482 | 0.1978 | 0.8519 | 1.0042 |
| methylsulfate | | | | |
| B: 1-ethyl-3-methylimidazolium | 0.1482 | 0.2216 | 0.7598 | 0.8533 |
| (EMIM) bromide | | | | |
| C: Choline acetate | 0.1482 | 0.2559 | 0.9480 | 1.0385 |
| D: 1-allyl-3 -methylimidazolium | 0.1482 | 0.2265 | 0.7091 | 0.8440 |
| (AMIM) chloride | | | | |
| E: Choline salicylate | 0.1482 | 0.1523 | 0.1542 | 0.1539 |
| F: Choline chloride | 0.1482 | 0.2445 | 1.0236 | 1.0674 |
| G: Tris(2-hydroxyethyl) | 0.1482 | 0.2605 | 0.8415 | 0.9087 |
| methylammonium methylsulfate | | | | |
| H: EMIM ethylsulfate | 0.1482 | 0.2499 | 0.7514 | 0.7965 |
| I: EMIM acetate | 0.1482 | 0.2324 | 0.7942 | 0.8223 |
| J: 1-butyl-3-methylimidazolium | 0.1482 | 0.2373 | 0.5994 | 0.8244 |
| (BMIM) bromide | | | | |
| DI (distilled water) | 0.1482 | 0.3000 | 1.0997 | 1.2130 |
| Mouthwash with 0.075% CPC | 0.1482 | 0.1706 | 0.1540 | 0.1704 |

It can also be seen from the above results that the anion of the choline-containing ionic liquid appears to play a significant role in imparting this growth inhibition activity, with salicylate providing the greatest antibacterial activity.

### Example 17

### Formulation of Ionic Liquid in a Chewing Gum

Xylitol, gum base, isomalt, gum arabic, flavors, maltitol syrup, titanium dioxide, coloring, shellac, carnauba wax, BHT (butylhydroxytoluene; to preserve freshness), and ionic liquid.

Whilst particular embodiments of the invention have been illustrated and described, it will be obvious to those skilled in the art that various changes and modifications may be made without departing from the scope of the invention as defined in the appended claims.

We claim:

1. An oral care composition comprising a therapeutically effective amount of at least one ionic liquid for use in removing or reducing plaque from the oral cavity of a subject and an orally acceptable carrier for a mouth rinse, toothpaste, oral beads or strips, irrigation fluid, plaque removal liquid, tongue spray, dental floss, candy, lozenge, chewing gum, patches and lollipop, wherein the ionic liquid is present in the composition in an amount of 5 wt % to 15 wt % based on the total weight of the composition,
wherein the ionic liquid comprises:
a) a heterocyclic cation is selected from the group consisting of pyrrolium, pyrrolinium, pyrrolidinium, oxazolium, thiazolium, imidazolium, imidazolidinium, pyrazolium, pyrazolinium, pyrazolidinium, isoxazolium, isothiazolium, oxadiazolium, triazolium, thiadiazolium, pyridinium, piperidinium, morpholinium, thiomorphilinium, pyridazinium, pyrimidinium, pyrazinium, piperazinium, and triazinium, or a quarternary ammonium cation of the formula 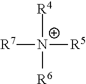
wherein R⁴, R⁵, R⁶ and R⁷ are each an organic moiety and may be the same or different, and at least one of R⁴, R⁵, R⁶ and R⁷ includes a hydroxyl, wherein R⁴, R⁵, R⁶ and R⁷ are independently selected from substituted or unsubstituted C₁₋₂₂ alkyl and C₂₋₂₂ alkenyl and at least one of R⁴, R⁵, R⁶ and R⁷ is substituted with a hydroxyl, and
b) an anion,
wherein if the ionic liquid comprises said heterocyclic cation the anion is selected from the group consisting of acetate, halide, phosphate, alkyl phosphate, phosphonate, pyrophosphate, hexametaphosphate, polymetaphosphate, orthophosphate, tripolyphosphate, sulfate, alkyl sulfate, lauryl sulfate, phenolsulfate. benzoate, acetylacetonate, carboxylate, citrate, ascorbate, dicyamide, L- or D-amino acids, glycolate, gluconate, maleate, sweetener anions, hydroxide, succinate, tartrate, docusate, linoleate, oleate, and tosylate,
and wherein if the ionic liquid comprises said quaternary ammonium cation, the anion is selected from the group consisting of salicylate, alkylsulfate, sulfate, acetate, halide, phosphate, alkyl phosphate and tosylate.

2. The oral care composition for use according to claim 1, wherein the heterocyclic cation is selected from the group consisting of imidazolium and pyrazolium.

3. The oral care composition for use according to claim 2, wherein the ionic liquid comprises:
a) an imidazolium cation of the formula 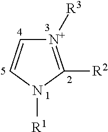
wherein
R¹, R² and R³ are independently selected from H, C₁₋₂₂ alkyl and C₂₋₂₂ alkenyl; and
b) an anion selected from the group consisting of acetate, halide, phosphate, alkyl phosphate, sulfate, alkyl sulfate, and tosylate.

4. The oral care composition for use according to claim 3, wherein
the ionic liquid is selected from the group consisting of 1-ethyl-3-methylimidazolium (EMIM) chloride, EMIM bromide, EMIM ethyl sulfate, EMIM diethyl phosphate, EMIM acetate, EMIM tosylate, 1-butyl-3-methylimidazolium (BMIM) chloride, BMIM bromide, BMIM methyl sulfate, BMIM octyl sulfate, BMIM acetate, 1-allyl-3-methylimidazolium (AMIN) chloride, 1-decyl-3-methylimidazolium (DMIM) chloride, and 1,2,3-trimethylimidazolium (TMIM) methyl sulfate.

5. The oral care composition for use according to claim 1, wherein R⁴, R⁵, R⁶ and R⁷ are independently selected from substituted or unsubstituted C₁₋₆ alkyl and C₂₋₆ alkenyl and at least one of R¹, R⁵, R⁶ and R⁷ is substituted with a hydroxyl.

6. The oral care composition for use according to claim 1, wherein the quaternary ammonium cation is choline or tris-(2-hydroxyethyl)methyl ammonium.

7. The oral care composition for use according to claim 6, wherein
the ionic liquid is selected from the group consisting of choline salicylate, tris-(2-hydroxyethyl)methylammonium methylsulfate and mixtures thereof.

8. The oral care composition for use according to claim 1, wherein the ionic liquid comprises:
a) a pyrazolium cation of the formula 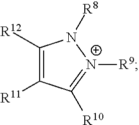
wherein,
R⁸, R⁹, R¹⁰, R¹¹ and R¹² are independently selected from H, C₁₋₂₂ alkyl and C₂₋₂₂ alkenyl; and
b) an anion selected from the group consisting of acetate, halide, phosphate, alkyl phosphate, sulphate, alkyl sulphate and tosylate.

9. The oral care composition of claim 8, wherein R⁸, R⁹, R¹⁰, R¹¹ and R¹² are independently selected from H and C₁₋₄ alkyl.

10. The oral care composition for use according to claim 9, wherein
the ionic liquid is 1,24-trimethylpyrazolium triethylsulfate.

11. The oral care composition for use according to claim 1, wherein the composition is substantially free of any abrasives.

12. The oral care composition for use according to claim 1, wherein the ionic liquid is present in the composition in an amount of 8 wt % to 10 wt % based on the total weight of the composition.

13. The oral care composition for use according to claim 1, wherein the composition further comprises one or more agents selected from diluents, bicarbonate salts, pH modifying agents, surfactants, foam modulators, thickening agents, viscosity modifiers, humectants, sweeteners, flavorants, pigments, anticaries agents, anticalculus or tartar control agents, and mixtures thereof.

14. (canceled)

15. A method of whitening teeth in a subject comprising administering to the subject an oral care composition comprising a therapeutically effective amount of an ionic liquid, wherein the oral care composition is as defined in claim 1.

16. (canceled)

17. A method of reducing the amount of bacteria, removing or reducing plaque from the oral cavity or reducing biofilm formation and/or degrading biofilm in the oral cavity of a subject comprising administering to the subject an oral care composition comprising a therapeutically effective amount of an ionic liquid, wherein the oral care composition is selected from a mouth rinse; toothpaste, toothpowder, oral bead or strip, fluid-encased dental strip, irrigation fluid, plaque removal liquid, dental floss, hard candy, soft candy, lozenge, chewing gum, or lollipop and
wherein the oral care composition is as defined in claim 1.

18.-21. (canceled)
